(19) United States
(12) Patent Application Publication
(10) Pub. No.: US 2020/0251182 A1
(43) Pub. Date: Aug. 6, 2020

(54) COGNITIVE IDENTIFICATION OF PATHOGENIC PATHWAYS

(71) Applicant: International Business Machines Corporation, Armonk, NY (US)

(72) Inventors: Daniel Enoch Platt, Putnam Valley, NY (US); ALDO GUZMAN SAENZ, White Plains, NY (US); Laxmi Parida, Mohegan Lake, NY (US); Subrata Saha, Ossining, NY (US)

(21) Appl. No.: 16/266,733

(22) Filed: Feb. 4, 2019

(51) Int. Cl. G16B 40/00; G06N 20/00; G16B 20/00; C12Q 1/6883; G16B 30/10

(52) U.S. Cl. CPC G16B 40/00; G06N 20/00; G16B 30/10; C12Q 1/6883; G16B 20/00

(57) ABSTRACT

Embodiments of the present invention are directed to methods for adapting machine learning, redescription, and computational homology techniques to the identification of pathogenic pathways. A non-limiting example of the computer-implemented method includes receiving genetic and biological data and generating a data matrix based on the data. The data matrix can include one or more features, and each feature can be associated with a known feature value. A collection of sets of features representing pathways, genes, or a genetic combination of genotype values can be determined. The method also includes determining a first prediction for a feature value of a selected feature to be predicted in the collection, permuting one or more rows of the data matrix, and recalculating a second prediction for the feature value based on the permutation. A prediction score can be determined based on the first prediction, the second prediction, and a known feature value.

### BACKGROUND

The present invention generally relates to computing systems and methods, and more specifically, to cognitive computing systems and methods configured to apply a novel configuration of machine learning, redescription, and computational homology techniques to the identification of pathogenic pathways.

Over the past decade, the development of industrial high-throughput genotyping platforms has led to large-scale genome-wide association studies (GWAS) and to the proliferation of readily available genetic and biological system data. Today, pathway analysis algorithms and bioinformatics systems can use this data to analyze biological pathways as part of the treatment of diseases. A biological pathway is an ordered set of interactions between intracellular molecules having collective activity that impacts cellular function, for example, by controlling metabolite synthesis or by regulating the expression of sets of genes. Pathway analysis algorithms and systems leverage various algorithms to analyze different omics data, such as transcriptomics, proteomics with protein—protein interactions, and metabolomics. Pathway-based analysis algorithms can be generally classified into one of four groups: over-representation analysis, gene set-based scoring, multivariate approaches, and topological-based analysis.

### SUMMARY

Embodiments of the present invention are directed to a computer-implemented method for adapting machine learning, redescription, and computational homology techniques to the identification of pathogenic pathways. A non-limiting example of the computer implemented method includes receiving genetic and biological data and generating a data matrix based on the data. The data matrix can include one or more features, and each feature can be associated with one or more known feature values. A collection of sets of features representing pathways, genes, or a genetic combination of genotype values can be determined. The method also includes determining a first prediction for a feature value of a selected feature to be predicted in the collection, permuting one or more rows of the data matrix, and recalculating a second prediction for the feature value based at least in part on the permutation. A prediction score can be determined based on the first prediction, the second prediction, and a known feature value.

Embodiments of the present invention are directed to a computer program product for adapting machine learning, redescription, and computational homology techniques to the identification of pathogenic pathways. A non-limiting example of the computer program product includes a computer readable storage medium readable by a processing circuit and storing program instructions for execution by the processing circuit for performing a method. The method includes receiving genetic and biological data and generating a data matrix based on the data. The data matrix can include one or more features, and each feature (e.g., SNP) can be associated with one or more known feature values (e.g. alleles). A collection of sets of features representing pathways, genes, or a genetic combination of genotype values can be determined. The method also includes determining a first prediction for a feature value of a selected feature to be predicted in the collection, permuting one or more rows of the data matrix, and recalculating a second prediction for the feature value based at least in part on the permutation. A prediction score can be determined based on the first prediction, the second prediction, and a known feature value.

Embodiments of the invention are directed to a processing system for adapting machine learning, redescription, and computational homology techniques to the identification of pathogenic pathways. The processing system can include a processor in communication with one or more types of memory. The processor can be configured to receive genetic and biological data. The processor can also be configured to generate a data matrix based on the data. The data matrix can include one or more features, and each feature can be associated with one or more known feature values. A collection of sets of features representing pathways, genes, or a genetic combination of genotype values can be determined. The processor can be further configured to determine a first prediction for a feature value of a selected feature to be predicted in the collection, permute one or more rows of the data matrix, and recalculate a second prediction for the feature value based at least in part on the permutation. A prediction score can be determined based on the first prediction, the second prediction, and a known feature value.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a block diagram illustrating one example of a processing system for practice of the teachings herein;

FIG. 2 depicts a flow diagram of a method according to one or more embodiments of the present invention;

FIG. 3 depicts a flow diagram of a method according to one or more embodiments of the present invention; and

FIG. 4 depicts an exemplary system according to one or more embodiments of the present invention.

The diagrams depicted herein are illustrative. There can be many variations to the diagram or the operations described therein without departing from the spirit of the invention. For instance, the actions can be performed in a differing order or actions can be added, deleted or modified.

In the accompanying figures and following detailed description of the described embodiments, the various elements illustrated in the figures are provided with two or three-digit reference numbers. With minor exceptions, the leftmost digit(s) of each reference number correspond to the figure in which its element is first illustrated.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” can be understood to include any integer number greater than or equal to one, i.e. one, two, three, four, etc. The terms “a plurality” can be understood to include any integer number greater than or equal to two, i.e. two, three, four, five, etc. The term “connection” can include both an indirect “connection” and a direct “connection.”

The terms “about,” “substantially,” “approximately,” and variations thereof, are intended to include the degree of error associated with measurement of the particular quantity or dimension based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Turning now to an overview of technologies that are more specifically relevant to aspects of the invention, the availability of genetic and other biological system data has increased exponentially over the past decade. Genetic data can be organized several different ways including, for example, around pathways, systems, gene-gene interactions including epistatic interactions, gene-environment interactions including metabiome interactions, and environment-driven epigenetic changes. Evidence of complex associations and causative factors, such as gene-gene and gene-environment interactions, can exist within the large sets of genetic and biological systems data currently available. Complex diseases can involve gene-environment interactions that could be identified within a relevant set of data including the pertinent genetic and environmental clues. Such interactions, however, could in some cases require relatively large genetic and environmental data sets for the relevant association to arise. The availability of genetic and other biological system data is increasing and, along with it, the potential to discover and interpret complex diseases and the basis for such diseases.

Examining and comparing genetic information and phenotypes across large populations can potentially reveal useful information for the treatment and identification of disease patterns, causes, and risks. For example, numerous genome-wide association studies (GWAS) of diseases have suggested thousands of causal and correlative links between DNA sequence variants and specific disease phenotypes. These suspected or validated variants often reside within genes and open reading frames that can be mapped to biological pathways.

GWAS can be used, for instance, to examine one or more genetic variants in a set of individuals to determine whether the variants are associated with particular traits. GWAS, for example, can potentially identify associations between genetic variants, such as single nucleotide polymorphisms (SNPs), and traits associated with human diseases or disorders. The types of information revealed by GWAS, however, can be limited. For instance, although GWAS can sometimes identify SNPs associated with traits in major human disease based on observable phenotype, GWAS can fail to reveal other types of relationships such as relevant interactions among a subset of SNPs.

The large quantities of genetic and biological systems data available and involved in analyzing relevant interactions and associations poses challenges to revealing relevant and useful patterns and interactions. Mining tools have potential to reveal evidence of relevant genetic associations and relationships. However, the large quantities of available data, and the large quantities of data that can be required to reveal complex relationships, pose challenges and can render conventional mining techniques unfeasible or inadequate.

Discriminant approaches, such as Linear Discriminant Analysis (LDA), can seek to determine a linear combination of features associated with an event or other variable. In context of genetic data analysis, for instance, LDA could potentially identify linear combinations of genetic variants, gene expression results, and other biological data that maximize the variance between groups marked by composite phenotypes. However, discriminant approaches can lead to relatively large increases in data volume, which can be problematic and/or unworkable in the context of large initial data sets such as those associated with genome-wide genetic analysis. Thus, determining genetic basis for metabolic pathways or compound phenotypes in large genetic and biological systems data sets with conventional discriminant approaches can be unworkable. Thus, while conventional systems could sometimes associate an observable phenotype, such as hypertension, with a subset of SNPs from principal component analysis (PCA) of a limited data set, identification of complex associations that might be revealed in a larger set of genetic and biological systems data, such as the genetic basis for metabolic pathways, phenotypes associated with combinations of SNPs, or the relationships between genetic variation, expression variation, epigenetic changes, and epistasis with disease, can be unattainable.

Turning now to an overview of the aspects of the invention, one or more embodiments of the invention address the above-described shortcomings of the prior art by providing cognitive computing systems programmed with cognitive computing algorithms configured to identify relationships among genetic data to identify redescription associations between genetic variation, expression variation, epigenetic changes, and epistasis with disease. In some embodiments of the invention, the cognitive computing algorithms can analyze genetic and biological systems data to evaluate complex diseases. In embodiments of the invention, the cognitive computing algorithms can include the use of PCA to facilitate discriminant analysis, such as LDA, to identify relevant genetic variants, such as SNPs, and/or their combinations, from relatively large data sets. In embodiments of the invention, the cognitive computing algorithms can generate relevant genetic variants associated with complex diseases and phenotypes by including pattern discovery to identify significant patterns, identifying significant redescriptions within the patterns, and then applying a discriminant analysis to reveal important discriminant variants.

In some embodiments of the invention, the cognitive computing system programmed with cognitive computing algorithms configured to extract information about how much a candidate pathway or gene system contributes to disease predictivity ascertained from a machine-learning algorithm. In some embodiments of the invention, the cognitive computing system can extract this information for submission to downstream redescription mining and computational homology systems and processes.

The cognitive computing system can be configured to receive genetic and biological systems data, including relatively large data sets, and to evaluate these data sets for gene-gene and gene-environment interactions and other higher order associations. In some embodiments of the invention, the cognitive computing system can be configured to characterize and identify SNPs associated with disease. In embodiments of the invention, the cognitive computing algorithms can determine a genetic basis for metabolic pathways, and the cognitive computing algorithms can utilize subsets of identified discriminating SNPs to provide a smaller data set to mine for significance. In some embodiments of the invention, the identified discriminating SNPs can be further evaluated with GWAS studies to further characterize genetic associations. The cognitive computing algorithms in accordance with embodiments of the invention can advantageously boost the determined significance of SNPs, leading to increased reliability in genetic association identifications. Some embodiments of the invention can enhance GWAS studies to provide greater specificity among leading genes.

The aforementioned results can be achieved by configuring the cognitive computing system in part as a classifier to provide a best prediction of disease based on genetic variants, expression variations, epigenetic changes, and epistasis, together with feature selection, if appropriate to the method, of individual subjects. To test the contribution of a pathway, gene system, or gene to the prediction, the cognitive computing system randomly shuffles the genetic variants, expression variations, epigenetic changes, or epistasis in the pathway, gene system, or gene. The cognitive computing system then recomputes the pathway, gene system, or gene's best prediction for individual subjects and compares this prediction to the pre-shuffled prediction. This shuffling procedure can be repeated multiple times by the cognitive computing system to assess the range available by random variation.

The cognitive computing system can be configured to determine whether the best predictions with unshuffled variants lies within the statistical variation measured by the shuffled variants. In some embodiments of the invention, the cognitive computing system recodes the predictions to identify whether shuffling the pathway, gene system, or gene reduced predictive accuracy, left it unchanged, or corrected the prediction with unshuffled data. As used herein, the variants themselves can be referred to as “features,” while the values the variants can take (e.g., corrected, left unchanged, or reduced predictivity) and phenotypes (e.g. disease status) are called “feature values.”

Once the feature values are known (e.g., corrected, left unchanged, or reduced predictivity) for each pathway, gene system, or gene, the cognitive computing system identifies sets of individuals whose shuffling produced the recoded feature values. In addition, the cognitive computing system can identify combinations of these feature values, called “patterns,” that appear more or less common among the individuals (feature sets) than would be expected by chance from randomly sampling from a population with independent feature values.

In some embodiments of the invention, clusters of patterns, called redescriptions, that refer to approximately the same feature sets, are identified by the cognitive computing system. In some embodiments of the invention, the cluster approximation can be characterized by a distance between sets, such as a Jaccard distance. From those equivalent patterns, logical inferences between features, and the individuals that demonstrate the relationships, can be identified.

In some embodiments of the invention, the distances computed by the cognitive computing system serve as input to standard computational homology algorithms, where the patterns are mapped to nodes, with a distance matrix serving to define the filtration.

In some embodiments of the invention, the cognitive computing system can perform PCA and singular value decomposition (SVD) with descriptors or compound phenotypes to identify an orthogonal basis that spans the sampled data space and provides a representation of the dataset in that basis. In some embodiments of the invention, the cognitive computing system can perform a discriminant analysis, such as LDA, to identify a discriminating subset of SNPs. Such methodologies are an improvement over conventional genome-wide assessments because they facilitate discriminant approaches that would otherwise be precluded because of the large volume of input data.

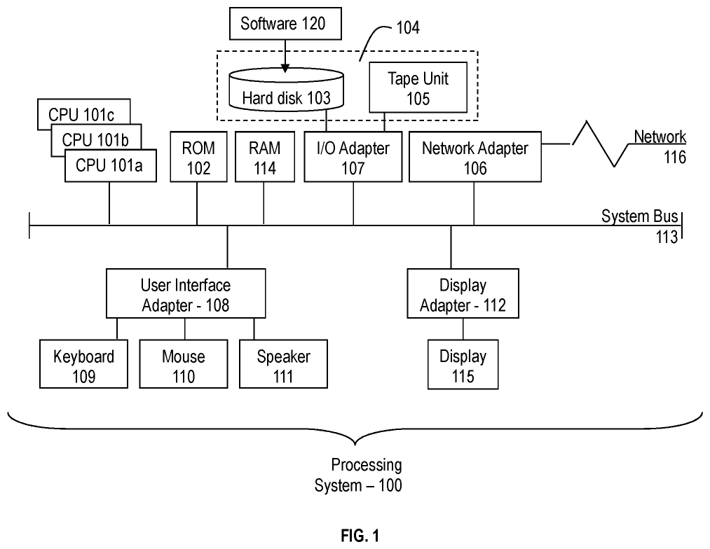

Turning now to a more detailed description of aspects of the invention, FIG. 1 depicts an embodiment of a processing system 100 for implementing the teachings herein. In this embodiment of the system 100, there is provided one or more central processing units (processors) 101a, 101b, 101c, etc. (collectively or generically referred to as processor(s) 101). In some embodiments of the invention, each processor 101 can include a reduced instruction set computer (RISC) microprocessor. Processors 101 are coupled to system memory 114 and various other components via a system bus 113. Read only memory (ROM) 102 is coupled to the system bus 113 and can include a basic input/output system (BIOS), which controls certain basic functions of system 100.

FIG. 1 further depicts an input/output (I/O) adapter 107 and a network adapter 106 coupled to the system bus 113. I/O adapter 107 can be a small computer system interface (SCSI) adapter that communicates with a hard disk 103 and/or tape storage drive 105 or any other similar component. I/O adapter 107, hard disk 103, and tape storage device 105 are collectively referred to herein as mass storage 104. Operating system 120 for execution on the processing system 100 can be stored in mass storage 104. A network adapter 106 interconnects bus 113 with an outside network 116 enabling data processing system 100 to communicate with other such systems. A screen (e.g., a display monitor) 115 is connected to system bus 113 by display adaptor 112, which can include a graphics adapter to improve the performance of graphics intensive applications and a video controller.

In some embodiments of the invention, adapters 106, 107, and 112 can be connected to one or more I/O busses that are connected to system bus 113 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 113 via user interface adapter 108 and display adapter 112. A keyboard 109, mouse 110, and speaker 111 all interconnected to bus 113 via user interface adapter 108, which can include, for example, a Super I/O chip integrating multiple device adapters into a single integrated circuit.

In some embodiments of the invention, the processing system 100 includes a graphics processing unit 130. Graphics processing unit 130 is a specialized electronic circuit designed to manipulate and alter memory to accelerate the creation of images in a frame buffer intended for output to a display. In general, graphics processing unit 130 is very efficient at manipulating computer graphics and image processing, and has a highly parallel structure that makes it more effective than general-purpose CPUs for algorithms where processing of large blocks of data is done in parallel.

Thus, as configured in FIG. 1, the system 100 includes processing capability in the form of processors 101, storage capability including system memory 114 and mass storage 104, input means such as keyboard 109 and mouse 110, and output capability including speaker 111 and display 115. In some embodiments of the invention, a portion of system memory 114 and mass storage 104 collectively store an operating system such as the AIX® operating system from IBM Corporation to coordinate the functions of the various components shown in FIG. 1.

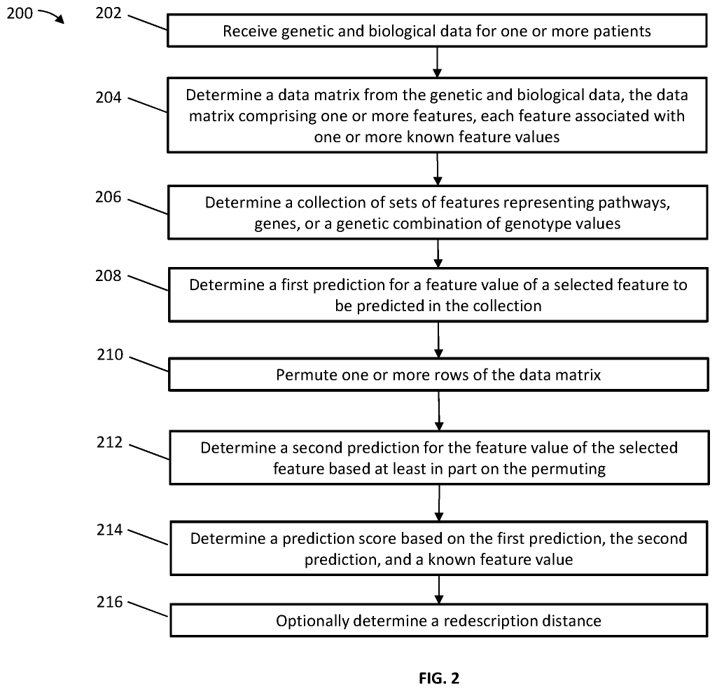

Continuing with the more detailed description of aspects of the present invention, FIG. 2 depicts a method 200 for evaluating the predictive accuracy of a candidate pathway or gene system (e.g., genetic variation, expression variation, epigenetic change, or epistasis) with respect to a disease, according to embodiments of the invention. The method 200 includes, as shown at block 202, receiving genetic and biological data for one or more patients. In some embodiments of the invention, the genetic and biological data includes a set of observations indexed by sample labels S.

The genetic data can include genomic or metagenomic data for an individual, for a subset of individuals, or for one or more populations including, for example, genetic variant data, expression variation data, epigenetic change data, epistasis data, and/or single nucleotide polymorphism data. Biological data can include, for example, medical data, such as medical diagnoses, medical history, and medication lists, phenotypes, demographic data, environmental data concerning a biological system, biological pathway data, metabolic data, biochemical data, or any other data that can potentially be correlated with a biological or genetic condition or state. The genetic and biological data can include data entries, such as “subjects” that include study subjects or enrollees in a study, “conditions” such as hypertension, diabetes or coronary artery disease, and the like.

The method 200 can also include, as shown at block 204, determining a data matrix X from the genetic and biological data received at block 202. In some embodiments of the invention, X is defined by rows having observations O and columns having features F. In some embodiments of the invention, each feature f∈F has an alphabet (set) of possible feature values V_{f}. In some embodiments of the invention, X includes a matrix of genotype and/or phenotype values (feature values) for each patient (observation). For example, X can include disease condition states for one or more subjects or patients labeled by s∈S. In some embodiments of the invention, X is a subset, specifically, X⊆S×(x_{f∈F}V_{f}).

The method 200 can also include, as shown at block 206, determining a collection P of sets of features F representing pathways, genes, or other genetic combinations of genotype values. In some embodiments of the invention, P⊆2^{F} where 2^{F} is the power set of all subsets of F. For example, P could be a collection of sets of SNPs, each set representing a pathway, by way of mapping SNPs to genes, and genes to pathways.

The method 200 can also include, as shown at block 208, determining a first prediction for a feature value of a selected feature to be predicted of one or more features in the collection P of the data matrix X In some embodiments of the invention, a classifier C is defined that produces a predicted feature value for one or more values in the data matrix X In other words, the classifier C can produce, for some selected feature f∈F, a predicted feature value u∈V_{f} for each of the labels s∈S. For example, C could be a linear SVM classifier trained on the current data to predict a column in X. In this manner, C produces a “best prediction” of disease based on genetic variants, together with feature selection if appropriate to the method, of individual subjects.

The method 200 can also include, as shown at block 210, permuting (also referred to as shuffling, or row shuffling) one or more rows of the data matrix X In some embodiments of the invention, this row permutation is random. In some embodiments of the invention, a permutation transformation Tₚ is defined for randomly shuffling, for each p∈P, the rows of each F∈p indexing columns in X. In some embodiments of the invention, Tₚ can be a different random permutation of the columns' values on columns found in p, and the identity map on all other columns. In some embodiments of the invention, permutation is done within available cases and controls.

The method 200 can also include, as shown at block 212, determining a second prediction for the feature value of the selected feature (or each of the one or more features) in the collection P of the data matrix X based on the random permutation obtained from block 210. In some embodiments of the invention, the classifier C is applied, for each p in P, to obtain k∈ [symbol] second predicted feature values u∈V_{f}, one from each application of Tₚ on X. This produces a set of data matrices {Xₚ_{₁₁}, . . . , Xₚ_{₁ₖ}, Xₚ_{₂₁}, . . . , Xₚ_{₂ₖ}, . . . , Xₚ_{ₙ₁}, . . . , Xₚ_{ₙₖ}}.

In some embodiments of the invention, Xₚᵢⱼ could be the result of randomly permuting genotype values for SNPs found in pathway p. In this manner, a null hypothesis is provided that represents multiple observations of random variation among associations between p and f. In other words, shuffling provides a way to test contributions to predictivity for each of the pathways p∈P that is independent of a machine learning algorithm (e.g., the classifier C), and in particular, preserves content of any non-linear associations due to epistasis that C could have identified.

In some embodiments of the invention, this initial prediction-shuffling-final prediction scheme can be repeated or otherwise replicated multiple times to assess a range of feature values available by random variation alone. In this manner, we can determine if the best predictions with unshuffled variants lies within the statistical variation measured by the shuffled variants.

The method 200 can also include, as shown at block 214, determining a prediction score based on the first prediction, the second prediction, and a known feature value (i.e., the stored feature value in the data matrix associated with the particular selected feature for which the first predicted feature value and second predicted feature value are made). The actual feature value is the known, or “true” feature value, against which the predicted feature values can be tested for accuracy.

In some embodiments of the invention, a prediction score w is determined for each p in P. The prediction score w can define the effect, if any, shuffling (block 210) had on the prediction calculated in block 212. In some embodiments of the invention, this operation can be denoted as CTₚX.

In some embodiments of the invention, the score w has a value of “0” (or “=”) if shuffling produced no change in the prediction of the actual feature value. In other words, the first predicted feature value and the second predicted feature value share the same predictive ability with respect to the actual feature value.

In some embodiments of the invention, the score w has a value of “1” (or “+”) if shuffling corrected a mismatch between the first predicted feature value and the actual feature value. In other words, after shuffling, the second predicted feature value provided a better prediction for the actual feature value (with respect to the first predicted feature).

In some embodiments of the invention, the prediction score w has a value of “2” (or “−”) if shuffling resulted in a loss of a correct prediction for the actual feature value. In other words, after shuffling, the second predicted feature value provided a worse prediction for the actual feature value (with respect to the first predicted feature).

In some embodiments of the invention, a single prediction score w for each sample s∈S is assigned from the k∈ [symbol] shufflings by majority. In other words, recoded feature values (e.g., corrected, left unchanged, or reduced predictivity) can be generated for each s∈S. In this manner, the predictive accuracy of a candidate pathway or gene system (e.g., genetic variation, expression variation, epigenetic change, or epistasis) with respect to a disease can be evaluated, and optionally, compared against other candidate pathways or gene systems.

The method 200 can also include, as shown at block 216, optionally determining a redescription distance. In some embodiments of the invention, the redescription distance is based on the recoded feature values.

For example, once the recoded feature values (e.g., corrected, left unchanged, or reduced predictivity) are known for each pathway, gene system or gene, sets of individuals whose shuffling produced the recoded feature values can be identified.

In some embodiments of the invention, combinations of these feature values, called “patterns,” that appear more or less common among the patients or individuals (feature sets) than would be expected by chance from randomly sampling from a population with independent feature values, are identified.

In some embodiments of the invention, clusters of patterns, called redescriptions, that refer to approximately the same sample sets, are identified. In some embodiments of the invention, this cluster approximation can be characterized by a distance between sets, such as, for example a Jaccard distance. From those “equivalent” patterns (i.e., patterns within some predetermined threshold, such as a Jaccard distance), logical inferences between features, and the individuals that demonstrate the relationships, can be identified.

In some embodiments of the invention, these distances (also referred to as redescription distances) serve as input to standard computational homology algorithms, where the patterns are mapped to nodes, with a distance matrix serving to define the filtration.

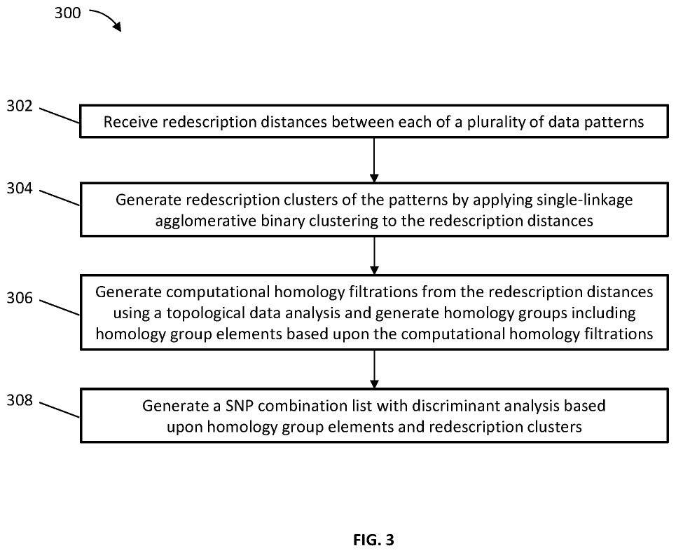

Continuing with the more detailed description of aspects of the present invention, FIG. 3 depicts a method 300 of genetic variant list generation according to embodiments of the invention. The method 300 includes, as shown at block 302, receiving redescription distances between each of a plurality of data patterns. In some embodiments of the invention, the redescription distances are received from upstream systems and/or processes. For example, the redescription distances can be determined as described previously herein (e.g., as described with respect to block 216 of FIG. 2).

The method 300 can also include, as shown at block 304, generating redescription clusters of the patterns by applying single-linkage agglomerative binary clustering to the redescription distances.

The method 300 can also include, as shown at block 306, generating computational homology filtrations from the redescription distances using a topological data analysis and generating homology groups including homology group elements based upon the computational homology filtrations.

The method 300 can also include, as shown at block 308, generating a genetic variant or SNP combination list based upon by the homology group elements and redescription clusters, including SNPs distinguished by the homology group elements and redescription clusters.

The method 300 optionally includes comparing the SNP combination list to GWAS-generated or GWAS-styled SNP combinations.

Redescriptions and redescription clusters, including statistically significant redescriptions and redescription clusters, can be identified within or generated from mined data, for example, through redescription mining. For example, data mining of medical data can generate a plurality of subsets of data including medical descriptors such as diabetes, hypertension, coronary heart disease, obesity, and cancer, to name a few. Redescription mining can include analyzing subsets of data associated with one or more descriptors that can be amenable to multiple different descriptions and generating appropriate redescriptions for such subsets based upon the analysis. Statistically significant redescriptions can identify composite phenotypes and logical relationships among phenotypes marking underlying biological processes associated by Jaccard or similar distances. Such biological processes can include, for instance, biological pathways, epistatic interactions, gene-environment interactions, and the like. Redescription can identify logical equivalence and construct logical relationships between variables.

Data mining can include any known method capable of selecting patterns according to statistical significance. Data mining can include pattern discovery methods, including selecting patterns according to statistical significance. Data mining can implement, for example, logistic regression. In some embodiments of the invention, data mining includes assigning a measure of significance to each pattern association, wherein the measure of significance quantifies the chance that a pattern association would be observed among samples as a result of random variation.

In some embodiments of the invention, the redescription distances are based upon a dissimilarity between lists of samples identified by each data pattern in the plurality of data patterns, for example as measured by Jaccard distances.

For example, given a set of data entries, patterns can be generated that reveal groups or subsets of subjects that share certain patterns (e.g., hypertension=T, diabetes=T, and coronary artery disease=F). Each such pattern can be associated with a list of data entries or subjects that share the pattern.

Distances, such as Jaccard distances, can be determined between such lists of data entries. In some embodiments of the invention, clusters can be defined based upon groups of items sharing at least one neighbor nearer than a given distance threshold. Such clusters can form equivalence classes, wherein all the patterns in a given cluster are assumed to be equivalent. For example, in the above exemplary scenario, if the “Diabetic=T” list and the “Diabetic=T and Hypertension=T” list are both in a cluster and have a high degree of similarity (i.e., few disagreements in their respective lists), then D=D and H such that D implies H (the diabetes list is a subset of the hypertension list). Such a result could reveal, for example, that diabetics tend to be hypertensive. Because, in the above exemplary scenario, Diabetes=T results in nearly the same list as Diabetes=T and Hypertension=T, both of these patterns capture the same list and can be said to “redescribe” that list. Equivalent patterns are referred to herein as “redescriptions.”

TDA computes homology groups generated through analysis of redescription distances among patterns. In some embodiments of the invention, TDA determines how the redescription clusters of patterns are related in topological space and can identify or distinguish between related and/or unrelated groups. Generation of homology groups can include identifying combinations in the mined data that are likely to be related for further analysis. In some embodiments of the invention, TDA is performed on calculated distances between lists of data entries sharing a pattern. In some embodiments of the invention, TDA can reveal relationships between and among patterns.

LDA computation can be performed by singular value decomposition (SVD), based on PCA with descriptors or compound phenotypes. SVD, for example, can recode the whole space spanned by the sample data, advantageously reducing the computational size of the mined data set in some embodiments of the invention, enabling downstream LDA analysis.

For example, redescription could reveal that most diabetics have hypertension. TDA can provide more information among related logical statements that redescription discovers, including some evidence of possible missing relationships (holes) in the data. These patterns can identify groups of data that share patterns (redescriptions), implying logical relationships among the patterns, and which form topological relationships among the patterns. Each of these groups of data represent augmented or compound phenotypes that reflect distinctive biological processes subject to the logical relationships among the data, e.g. “most type 2 diabetics have hypertension.” Such augmented phenotypes can then show distinct genetic relationships among them that can be probed using GWAS or LDA. This can give distinctive genetic signatures marking specific biological pathways marked by the redescription clusters.

Some embodiments of the invention include performing a discriminant analysis. Discriminant analysis can include, for example, LDA, support vector machine (SVM) approaches, or similar analysis. Discriminant analysis can be applied to homology groups alone or in combination with other data, such as redescription clusters. Discriminant analyses can be applied to homology groups, genetic data, gene expression data, and the like and can maximize the variation of linear combinations of genetic markers between versus within groups identified by the redescription clusters and homology groups. In some embodiments of the invention, discriminant analysis yields a genetic variant list including linear combinations, most important discriminant variants, and/or statistical significance for the linear combinations, such as p-values. In some embodiments of the invention, linear combinations can include SNP or gene combinations such as gene-gene combinations, gene-environment combinations, or gene-phenotype combinations.

Some embodiments of the invention include performing GWAS or a GWAS-style measure. Methods of performing such studies and measures are known. For example, one embodiment of the invention can include a genome wide comparison of a healthy set of individuals to a subset of individuals having a certain characteristic, such as a disease. In some embodiments of the invention, GWAS includes analysis of linear combinations from an SNP combination list generated in a discriminant analysis.

In some embodiments of the invention, GWAS is performed on the same data set used to generate an SNP combination list according to embodiments of the invention and an SNP list generated by the GWAS analysis is compared to the SNP combination list. Based upon the comparison, some embodiments of the invention can include generating a refined SNP list including, for example, a subset of the SNP combination list that is also included on the SNP list generated by the GWAS analysis.

In this manner, embodiments of the invention can identify redescription associations between genetic variation, expression variation, epigenetic changes, and epistasis with disease. Moreover, embodiments of the invention can identify connections between phenotypes and multiple SNPs or other genetic variants.

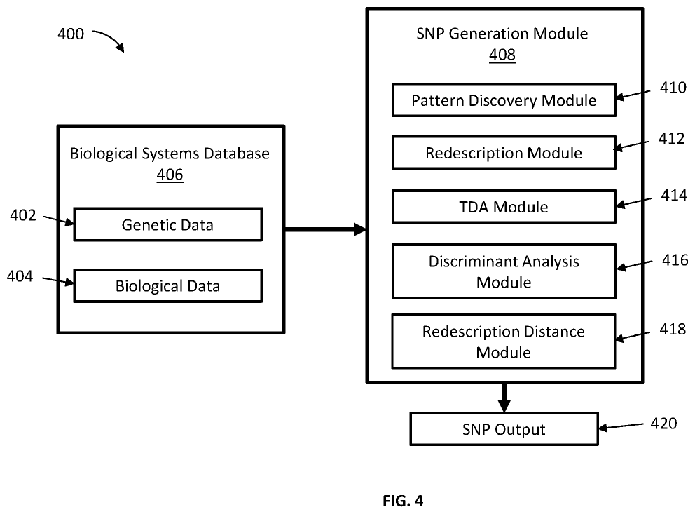

FIG. 4 depicts an exemplary system 400 for SNP identification. The system 400 can include a biological systems database 406 including, for example, genetic data 402 and biological data 404. The system 400 can also include an SNP generation module 408 in communication with the biological systems database 406.

In some embodiments of the invention, the SNP generation module 408 can include a pattern discovery module 410. The pattern discovery module 410 can mine genetic and biological data to identify statistically significant patterns, according to one or more embodiments.

The SNP generation module 408 can also include a redescription module 412. The redescription module 412 can, for instance, identify statistically significant redescriptions within the data to generate logical relationships between genetic variants, expression variants, epigenetic changes, and epistasis with disease as associated by Jaccard distances.

The SNP generation module 408 can also include a TDA module 414. The TDA module 414 can, for example, construct homology groups from the Jaccard distances generated by the redescription module 412.

The SNP generation module 408 can also include a discriminant analysis module 416. The discriminant analysis module 416 can apply a discriminant approach, including LDA, GWAS, and/or other discriminant methods, to the data to generate individual, or linear combinations of the most important discriminant variants, and to quantify significance, according to one or more embodiments.

The SNP generation module 408 can also include a redescription distance module 418. The redescription distance module 418 can determine one or more redescription distances between each of a plurality of data patterns, according to one or more embodiments. In some embodiments of the invention, the redescription distance module 418 determines, generates, or receives one or more redescription distances in a similar manner as described with respect to step 216 of FIG. 2. In some embodiments of the invention, the redescription distance module 418 provides one or more of these redescription distances to the SNP Generation Module 408 and/or the redescription module 412.

The system 400 can also include an SNP output 420. The SNP output 420 can include, for instance, a user interface, such as a display, for providing linear combinations, most important discriminant variants, significance and/or related data generated by the SNP Generation module 408 to a user.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may include copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages, languages supported on virtual environments such as Java or Python, or interpreted computational environments, such as R. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which includes one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments described. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method comprising:
receiving, by a processor, genetic data and biological data;
determining, by the processor, a data matrix from the genetic and biological data, the data matrix comprising one or more features, each feature associated with one or more known feature values;
determining, by the processor, a collection of sets of features, the collection representing pathways, genes, or a genetic combination of genotype values;
determining, by the processor, a first prediction for a feature value of a selected feature to be predicted in the collection;
permuting, by the processor, one or more rows of the data matrix;
determining, by the processor, a second prediction for the feature value of the selected feature based at least in part on the permuting; and
determining, by the processor, a prediction score based on the first prediction, the second prediction, and a known feature value of the selected feature.

2. The computer-implemented method of claim 1, wherein the prediction score comprises a first value if the first prediction of the feature value and the second prediction of the feature value are equal, a second value if the first prediction of the feature value agrees with the known feature value and the second prediction of the feature value disagrees, and a third value if the second prediction of the feature value agrees with the known feature value and the first prediction of the feature value disagrees with the known feature value.

3. The computer-implemented method of claim 1, wherein the data matrix comprises genotype values for one or more patients.

4. The computer-implemented method of claim 1, wherein the feature values comprise genotype values or phenotype values for one or more patients.

5. The computer-implemented method of claim 1, wherein the collection comprises sets of single nucleotide polymorphisms (SNPs).

6. The computer-implemented method of claim 1, wherein the second predicted feature value is determined after permuting the one or more rows of the data matrix.

7. The computer-implemented method of claim 1 further comprising determining, by the processor, a redescription distance based on the prediction score.

8. A computer program product comprising a computer readable storage medium readable by a processing circuit and storing program instructions for execution by the processing circuit for performing a method comprising:
receiving genetic data and biological data;
determining a data matrix from the genetic and biological data, the data matrix comprising one or more features, each feature associated with one or more known feature values;
determining a collection of sets of features, the collection representing pathways, genes, or a genetic combination of genotype values;
determining a first prediction for a feature value of a selected feature to be predicted in the collection;
permuting one or more rows of the data matrix;
determining a second prediction for the feature value of the selected feature based at least in part on the permuting; and
determining a prediction score based on the first prediction, the second prediction, and a known feature value of the selected feature.

9. The computer program product of claim 8, wherein the prediction score comprises a first value if the first prediction of the feature value and the second prediction of the feature value are equal, a second value if the first prediction of the feature value agrees with the known feature value and the second prediction of the feature value disagrees, and a third value if the second prediction of the feature value agrees with the known feature value and the first prediction of the feature value disagrees with the known feature value.

10. The computer program product of claim 8, wherein the data matrix comprises genotype values for one or more patients.

11. The computer program product of claim 8, wherein the feature values comprise genotype values or phenotype values for one or more patients.

12. The computer program product of claim 8, wherein the collection comprises sets of single nucleotide polymorphisms (SNPs).

13. The computer program product of claim 8, wherein the second predicted feature value is determined after permuting the one or more rows of the data matrix.

14. The computer program product of claim 8 further comprising determining a redescription distance based on the prediction score.

15. A processing system comprising:
a processor in communication with one or more types of memory, the processor configured to:
receive genetic data and biological data;
determine a data matrix from the genetic and biological data, the data matrix comprising one or more features, each feature associated with one or more known feature values;
determine a collection of sets of features, the collection representing pathways, genes, or a genetic combination of genotype values;
determine a first prediction for a feature value of a selected feature to be predicted in the collection;
permute one or more rows of the data matrix;
determine a second prediction for the feature value of the selected feature based at least in part on the permuting; and
determine a prediction score based on the first prediction, the second prediction, and a known feature value of the selected feature.

16. The processing system of claim 15, wherein the prediction score comprises a first value if the first prediction of the feature value and the second prediction of the feature value are equal, a second value if the first prediction of the feature value agrees with the known feature value and the second prediction of the feature value disagrees, and a third value if the second prediction of the feature value agrees with the known feature value and the first prediction of the feature value disagrees with the known feature value.

17. The processing system of claim 15, wherein the data matrix comprises genotype values for one or more patients.

18. The processing system of claim 15, wherein the feature values comprise genotype values or phenotype values for one or more patients.

19. The processing system of claim 15, wherein the collection comprises sets of single nucleotide polymorphisms (SNPs).

20. The processing system of claim 15, wherein the second predicted feature value is determined after permuting the one or more rows of the data matrix.
